(19) United States
(12) Patent Application Publication
(10) Pub. No.: US 2019/0224005 A1
(43) Pub. Date: Jul. 25, 2019

(54) Docking Device for Aortic Valve Replacement

(71) Applicant: Michael B. McDonald, Cordova, TN (US)

(72) Inventor: Michael B. McDonald, Cordova, TN (US)

(21) Appl. No.: 16/251,546

(22) Filed: Jan. 18, 2019

Related U.S. Application Data
(60) Provisional application No. 62/619,448, filed on Jan. 19, 2018.
(60) Provisional application No. 62/627,910, filed on Feb. 8, 2018.

(51) Int. Cl. A61F 2/24

(52) U.S. Cl. CPC A61F 2/2409; A61F 2210/0014

(57) ABSTRACT

A docking device for aortic valve replacements is a self-expanding nitinol structure including a lattice forming a tubular (e.g., cylindrical) upper framework or cage, and three struts forming a lower structure. The struts are formed by elongated loops that are attached to the lattice or that are an integral part of the upper framework. The struts extend down beneath the lattice, eventually forming “feet” to anchor at the base of the aortic cusps. The docking device can be positioned near a sinotubular junction of a patient. An aortic valve replacement is locked inside the docking device.

### CROSS-REFERENCE TO RELATED APPLICATIONS

This application claims the benefit of priority to U.S. provisional application Ser. No. 62/619,448 filed on Jan. 19, 2018, which is incorporated herein by reference.

This application also claims the of priority to U.S. provisional application Ser. No. 62/627,910 filed on Feb. 8, 2018, which is incorporated herein by reference.

### BACKGROUND

This disclosure relates generally to methods and apparatus for locking an aortic valve replacement near a sinotubular junction of a patient.

Most of the present percutaneous aortic valve replacements (also referred to as transcatheter aortic valves) are designed to be used in patients suffering from aortic stenosis. In almost all cases of aortic stenosis, there is a significant amount of calcium in the aortic valve. This deposition of calcium is the main feature that holds these aortic valve replacements in place in the aortic annulus.

Patients with aortic insufficiency usually do not have a significant amount of calcium deposited in the native aortic valve. This lack of calcium to grab the valve can result in difficulty properly positioning the transcatheter aortic valve replacement and holding it in place. Valve embolization is usually catastrophic.

Thus, there is a continuing need in the art for methods and apparatus for locking an aortic valve replacement near a sinotubular junction of a patient.

### BRIEF SUMMARY OF THE DISCLOSURE

The disclosure describes a docking device that may be positioned near a sinotubular junction of a patient. The docking device may be used for locking an aortic valve replacement.

The docking may comprise a lattice, which may be expandable. The lattice may form a tubular framework.

The docking may comprise a plurality of struts attached around the lattice. For example, the plurality of struts may comprise at least three struts. Each of the plurality of struts may comprise an upper or proximal part and a lower or distal part. The lower or distal part may comprise an elongated loop formed by a doubling of a wire. The doubling of the wire may form an inverted arch. The lower or distal part may not be attached to the lattice and/or may be shaped longitudinally as a curve protruding outward relative to the tubular framework. A lowermost end of the lower or distal part may be shaped transversely as a curve protruding inward relative to the tubular framework. The upper or proximal part may be attached to the lattice and/or shaped longitudinally as a curve protruding inward relative to the tubular framework. The curve may be flattening out toward an uppermost end of the upper or proximal part.

The disclosure describes a system that may be positioned near the sinotubular junction of the patient.

The system may comprise an aortic valve replacement and the docking device. The lattice of the docking device may comprise inner protrusions sized to interlock with the aortic valve replacement.

The disclosure describes a method for using the docking device.

The method may comprise positioning the docking device near the sinotubular junction of the patient. For example, the upper or proximal part of each of the plurality of struts may be positioned in an ascending aorta of the patient. The lower or distal part of each of the plurality of struts may be positioned in a Sinus of Valsalva of the patient. Preferably, the lowermost end of the lower or distal part of each of the plurality of struts may be rested in the base of the aortic cusps.

The method may comprise expanding the lattice of the docking device. For example, the lower or distal part of each of the plurality of struts may be expanded first. Then, the upper or proximal part of each of the plurality of struts may be further expanded by introducing the aortic valve replacement inside the docking device. Preferably, expanding the upper or proximal part of each of the plurality of struts may then cause the lower or distal part of each of the plurality of struts to move inward such that aortic valve leaflets are pinched between the docking device and the aortic valve replacement.

The method may comprise locking the aortic valve replacement inside the docking device, which may be performed using the inner protrusions of the lattice.

### BRIEF DESCRIPTION OF THE DRAWINGS

For a more detailed description of the embodiments of the disclosure, reference may now be made to the accompanying drawings, wherein:

FIG. 1 is a frontal view of a docking device;

FIG. 2 is a side view of one of the plurality of struts of the docking device shown in FIG. 1;

FIG. 3 is a frontal view of the strut shown in FIG. 2;

FIG. 4 is a sectional view of a lowermost end portion of the lower or distal part of the stmt shown in FIG. 2;

FIG. 5 is a frontal view of a lattice of the docking device shown in FIG. 1;

FIG. 6 is a schematic view of the docking device shown in FIG. 1, illustrated positioned near a sinotubular junction of a patient, before an aortic valve replacement has been locked inside the docking device; and

FIG. 7 is a schematic view of the docking device shown in FIG. 1, after the aortic valve replacement has been locked inside the docking device.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Finally, the exemplary embodiments presented below may be combined in any combination of ways, i.e., any element from one exemplary embodiment may be used in any other exemplary embodiment, without departing from the scope of the disclosure.

All numerical values in this disclosure may be approximate values unless otherwise specifically stated. Accordingly, various embodiments of the disclosure may deviate from the numbers, values, and ranges disclosed herein without departing from the intended scope. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

Certain terms are throughout the following description and claims refer to particular components. As one having ordinary skill in the art may appreciate, various entities may refer to the same component by different names, and as such, the naming convention for the elements described herein is not intended to limit the scope of the invention, unless otherwise specifically defined herein. Further, the naming convention used herein is not intended to distinguish between components that differ in name but not function.

Presently, the use of the aortic valve replacement for the treatment of aortic insufficiency has been limited due to the precarious challenge of anchoring the aortic valve replacement in a non-stenotic aortic valve that lacks significant calcification. A docking device 10 for aortic valve replacement as described herein may solve this problem by providing a new option of valve repair for patients with aortic insufficiency.

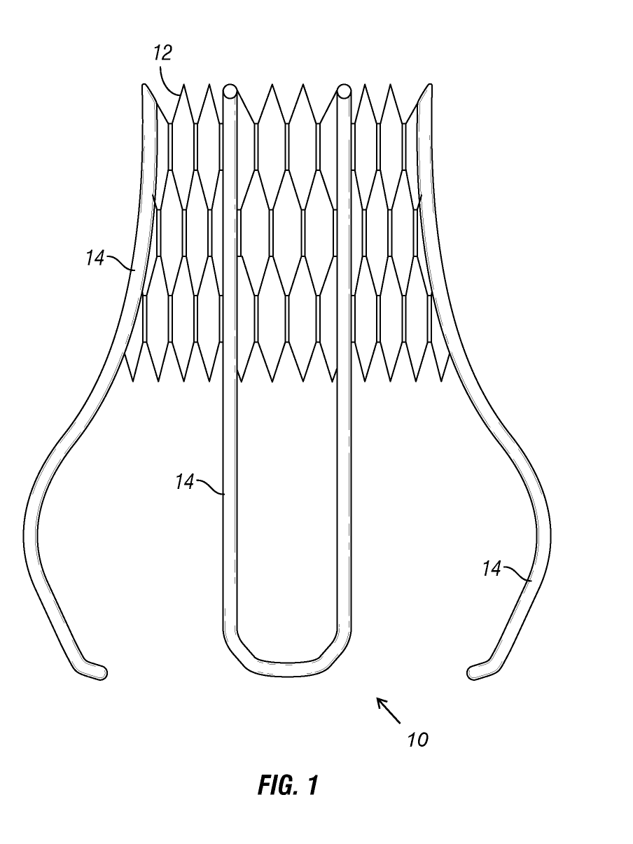

Referring to FIG. 1, the docking device 10 is a uniquely designed, self-expanding structure consisting of a lattice 12 forming a tubular (e.g., cylindrical) upper framework or cage, and three struts 14 extending down beneath the lattice 12, eventually forming a lower structure consisting of “feet.” The docking device 10 may be made of biocompatible material, preferably nitinol. The struts 14 are attached to the lattice 12 or are an integral part of the upper framework. The struts 14 are formed by elongated loops. The elongated loops are formed by a doubling of a wire. The doubling of the wire may form an inverted arch.

This self-expanding structure may be delivered as initially compressed on the distal end of a catheter. The delivery of the docking device 10 may be performed using an over the wire (wire guided) catheter in the 14-French diameter range. The catheter can include a main shaft in the 12-French diameter range. On the distal end of the main shaft, the docking device 10 can be compressed and held compressed by the outer sleeve that is over the main shaft. The outer sheath of the catheter may keep the self-expanding structure compressed on the main shaft of the catheter. The outer sleeve can be retracted or advanced from the distal end of the main shaft by rotation of a handle over the proximal end of the catheter. Control of the position of the outer sleeve allows expansion or contraction of the docking device 10 as well as the eventual release of the docking device 10 from the delivery catheter. In one embodiment, the docking device 10 may have tabs on the top of the upper frame to aid in securing the docking device 10 in the delivery system and the release of the docking device 10 from the delivery system.

When the distal end of the catheter is properly positioned in the Sinus of Valsalva above the aortic valve, the outer sheath may be slowly retracted. This sheath retraction allows expansion of the docking device 10 to begin. Slight forward advancement of the main catheter shaft as the docking device 10 expands off the distal end of the catheter may keep the lowest end 28 of the docking device 10 flush against the outer rim of the aortic valve. Thus the “feet” can be used to anchor at the base of the aortic cusps (i.e., the outer rim of the aortic valve).

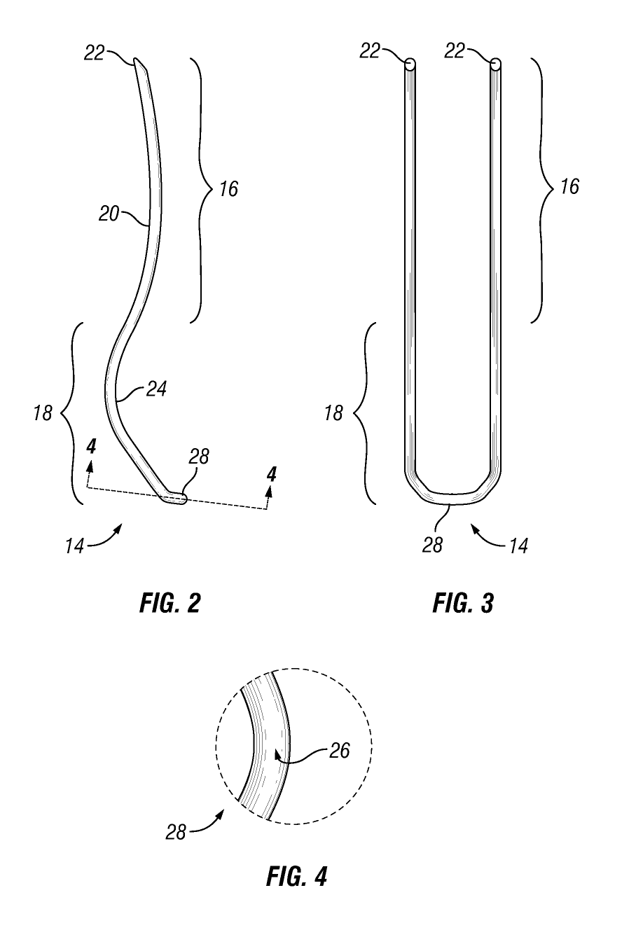

Referring to FIGS. 2 and 3, the lower or distal part 18 of the elongated loops of the docking device 10 have a concave (outward) shape 24. The lowest end 28 of the elongated loops may be positioned against the outer rim of the aortic valve where it joins of the walls of the Sinus of Valsalva. The rest of the lower or distal part 18 of the elongated loops may be abutting the upper walls of the Sinus of Valsalva just under the sinotubular junction of the ascending aorta. Thus, the lower or distal part 18 of the elongated loops may form a locked configuration along the walls of the Sinus of Valsalva. In other words, the distal contact points of the struts 14 against the aortic valve and the proximal contact points of the struts 14 against the roof of the Sinus of Valsalva may secure the docking device 10, thus preventing its movement in either direction.

The upper or proximal part 16 of the docking device 10 includes a slightly concave (inward) shape 20. The uppermost end 22 of the elongated loops of the docking device 10 flattens out. Note that the lattice 12 essentially conforms to the shape of the upper or proximal part 16. The middle portions of the struts 14 are curved to match the anatomy of the upper part of the Sinus of Valsalva near the sinotubular junction.

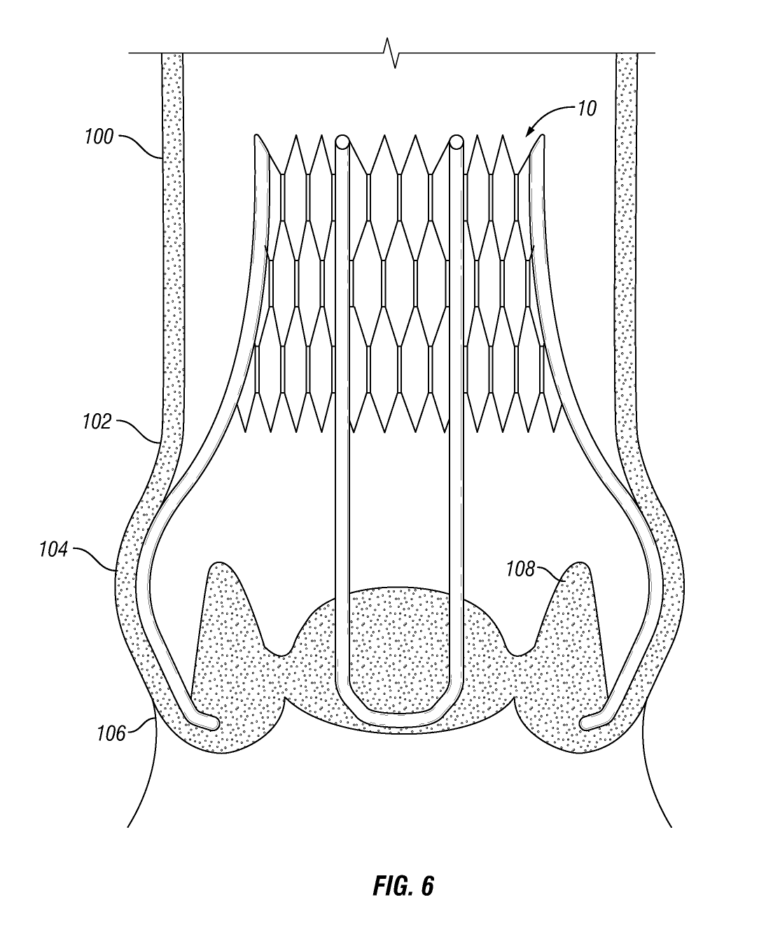

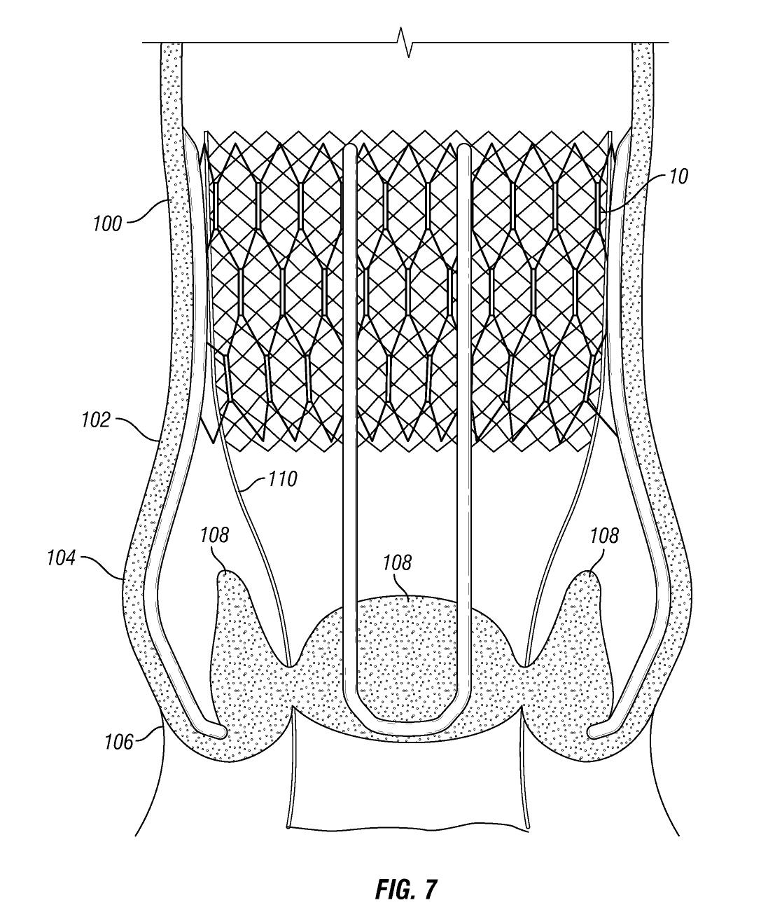

The lowest end 28, can include a slightly concave (inward) shape 26 (shown in the sectional view of FIG. 4) that may have a rough surface. Further, as a result of the shape and configuration of the docking device 10, when the upper part 16 of the docking device 10 is expanded outward, the lower part 18 may move inward, as illustrated in the sequence of FIGS. 6 and 7. Thus, the lowest end 28 of the elongated loops of the docking device 10 may pinch the aortic valve leaflets in between the docking device 10 and the aortic valve replacement. This position change of the struts 14 can create another locking area and may help in the sealing against paravalvular leaks around the aortic valve replacement.

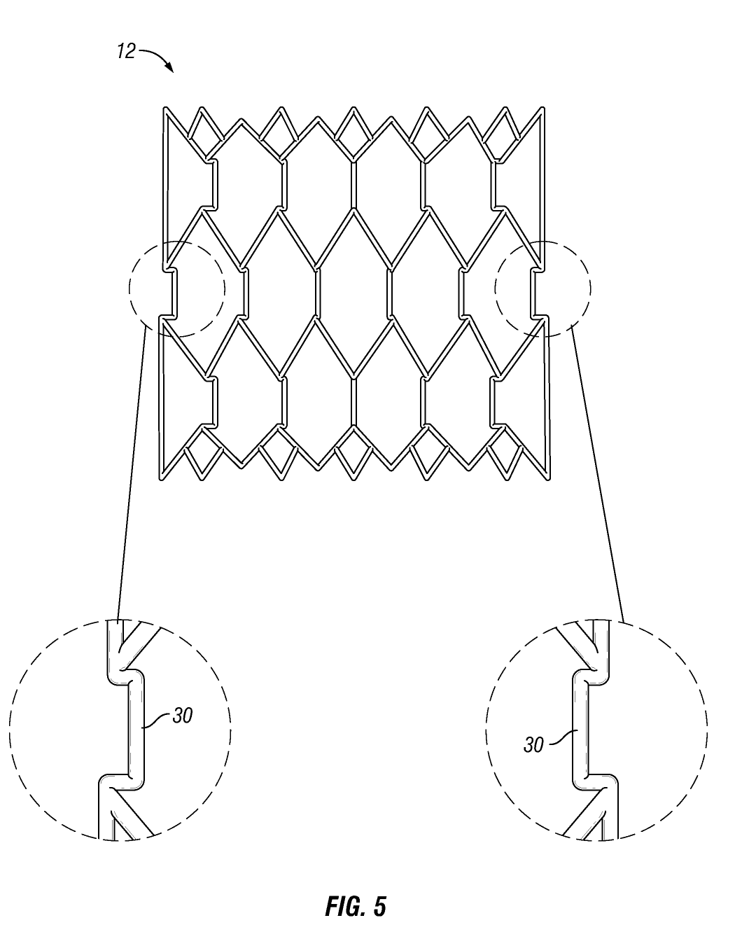

Referring to FIG. 5, the slightly concave (inward) middle part of the lattice 12 includes small catches. These small catches preferably include inward segments 30. These inward segments 30 may serve as locks into the structure of the aortic valve replacement. For example, as the aortic valve replacement expands during its deployment inside the docking device 10, as illustrated in FIG. 7, it may interlock against and further expand the docking device 10. Accordingly, the docking device 10 and the aortic valve replacement may lock together. The aortic valve replacement may thus be stabilized with the docking device 10, preventing vertical movement in either aortic or ventricular direction.

Referring to FIG. 6, the docking device 10 may be mounted on and over the wire of a device delivery catheter in the 14-French range for its deployment. The docking device 10 is compressed onto the distal end of the device delivery catheter while the outer sheath of the delivery catheter is advanced over the docking device 10 to keep it compressed on the catheter. The device delivery catheter may function in the same way some of the present delivery systems function in deploying aortic valve replacements. Once a guidewire is placed through the aortic valve into the left ventricle, the delivery catheter with the compressed docking device 10 is advanced on the guidewire over the aortic arch and into the Sinus of Valsalva 104. The distal end of the docking device 10 is advanced to the upper level of the Sinus of Valsalva 104. At this level, retraction of the outer sheath of the delivery catheter is begun.

The docking device 10 gradually starts to expand out from the delivery catheter as the sheath of the delivery catheter is retracted. The struts that make up the feet are incorporated as part of the upper framework of the docking device 10. As the docking device 10 flares out from the delivery catheter, the delivery catheter is advanced until the feet of the docking device 10 are positioned down into the aortic cusps at the bottom of the Sinus of Valsalva 104. Further retraction of the delivery catheter sheath continues to free the struts in the middle of the docking device 10.

The struts now expand against the walls and roof of the Sinus of Valsalva 104. The lower or distal part of the elongated loops of the docking device 10 serve as landing devices (or feet) to anchor the docking device 10 against the native aortic valve of a patient. Accordingly, the concave (outward) shape of the lower or distal parts of the elongated loops expands against the walls of the Sinus of Valsalva 104 to prevent upward movement of the docking device 10 away from the native aortic valve. Further, the lowermost end of the feet of the struts rests at the base of the aortic valve leaflets 108 in the aortic cusps preventing the docking device 10 from moving in the ventricular direction. Thus, the locking points of the docking device 10 to the patient are the middle curvature portion of the struts against the upper walls and roof of the Sinus of Valsalva 104 and the foot section of the struts against the base of the aortic valve leaflets 108. Prior to the full release of the docking device 10, a stable position (lock) can be confirmed by a gentle push/pull on the delivery catheter.

As the remainder of the sheath is retracted, the lattice 12 forming the upper cage of the docking device 10 is released at the level of the sinotubular junction and just above. After the release of the docking device 10, the delivery catheter is removed from the patient while the guidewire is left in place in the left ventricle. Once released, the upper cage does not fully expand against the walls of the aorta. Thus, the docking device 10 may be of slightly less diameter than the top section of the aortic valve replacement 110 after the aortic valve replacement 110 is expanded.

Referring to FIG. 7, the aortic valve replacement 110 is then advanced over the same guidewire and into proper position at the level of the aortic annulus. As the aortic valve replacement 110 is deployed in the same manner as the docking device 10 was, the lower part of the valve expands in the aortic annulus. Subsequently, the upper part of the aortic valve replacement 110 may expand inside the upper part of the docking device 10. The upper part of the aortic valve replacement 110 may include a nitinol structure that is stiffer and larger than the lattice of the docking device 10. This differential in stiffness may result in the aortic valve replacement 110 forcing the shape of the lattice of the docking device 10 after final expansion. Accordingly, the upper part of the aortic valve replacement 110 may cause some further expansion of the upper portion of the docking device 10.

Firstly, as the upper part of the docking device 10 is expanded by the aortic valve replacement 110, the lower part of the struts of the docking device 10 are moved inward forcing the feet against the aortic valve leaflets 108. The leaflets then become pushed against the aortic valve replacement 110. The docking device 10 feet, aortic valve leaflets 108, and aortic valve replacement 110 are now all pushed together. In other words, the feet may not only secure the docking device 10 to the aortic side of the native aortic valve, but they may move inward against the aortic valve leaflets 108 in the final deployment stage of the aortic valve replacement 110. This inward action results in a pinching point of the aortic valve leaflets 108 against the newly deployed aortic valve replacement 110.

Secondly, the upper structures of the docking device 10 and the newly deployed aortic valve replacement 110 are of complementary shape and may position layer to layer (interlocked). For example, the upper part of the docking device 10, which includes the lattice forming a cage, is designed to interlock with the upper structure of an aortic valve replacement 110. The main interlocking mechanism between the docking device 10 and the aortic valve replacement 110 are the inward protruding segments provided by of the lattice of the docking device 10. These inward protrusions interlock with the cells of the nitinol frame of the aortic valve replacement 110. This interaction occurs when the aortic valve replacement 110 expands against the upper part of the docking device 10. Because the uncompressed and unexpanded size of the lattice of the docking device 10 is less than the uncompressed and unexpanded size of the aortic valve replacement 110, the lattice of the docking device 10 may snuggly fit to the upper frame of the aortic valve replacement 110 after the final expansion the aortic valve replacement 110. Accordingly, the upper structure of nitinol cells of the aortic valve replacement 110 may interlock with the inner protrusions of the docking device 10 essentially permanently.

Once the aortic valve replacement 110 is released, the lower part of the aortic valve replacement 110 and docking device 10 are substantially locked together with contact between the feet of the docking device 10, aortic valve leaflets 108, and the aortic valve replacement 110 itself. The upper part of the docking device 10 and the upper part of the aortic valve replacement 110 are locked together by the inner protrusions of the docking device 10 positioning into the nitinol cells of the aortic valve replacement 110.

While the disclosure is susceptible to various modifications and alternative forms, specific embodiments thereof are shown by way of example in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

What is claimed is:

1. A docking device for positioning near a sinotubular junction of a patient and for locking an aortic valve replacement, the docking device comprising:
a lattice forming a tubular framework, wherein the lattice is expandable; and
a plurality of struts attached around the lattice.

2. The docking device of claim 1, wherein the plurality of struts comprises at least three struts.

3. The docking device of claim 1, wherein each of the plurality of struts comprises:
an upper or proximal part attached to the lattice; and
a lower or distal part that is shaped longitudinally as a curve protruding outward relative to the tubular framework.

4. The docking device of claim 3, wherein the upper or proximal part is shaped longitudinally as a curve protruding inward relative to the tubular framework and flattening out toward an upmost end of the upper or proximal part.

5. The docking device of claim 3, wherein the lower or distal part is not attached to the lattice.

6. The docking device of claim 5, wherein a lowermost end of the lower or distal part is shaped transversely as a curve protruding inward relative to the tubular framework.

7. The docking device of claim 3, wherein the lower or distal part comprises an elongated loop formed by a doubling of a wire, wherein the doubling of the wire forms an inverted arch.

8. A system for positioning near a sinotubular junction of a patient, the system comprising:
an aortic valve replacement; and
a docking device for locking the aortic valve replacement, wherein the docking device comprises a lattice forming a tubular framework and a plurality of struts attached around the lattice, wherein the lattice is expandable,
wherein the lattice comprises inner protrusions sized to interlock with the aortic valve replacement.

9. The system of claim 8, wherein the plurality of struts comprises at least three struts.

10. The system of claim 8, wherein each of the plurality of struts comprises:
an upper or proximal part attached to the lattice; and
a lower or distal part that is shaped longitudinally as a curve protruding outward relative to the tubular framework.

11. The system of claim 10, wherein the upper or proximal part is shaped longitudinally as a curve protruding inward relative to the tubular framework and flattening out toward an upmost end of the upper or proximal part.

12. The system of claim 10, wherein the lower or distal part is not attached to the lattice.

13. The system of claim 12, wherein a lowermost end of the lower or distal part is shaped transversely as a curve protruding inward relative to the tubular framework.

14. The system of claim 10, wherein the lower or distal part comprises an elongated loop formed by a doubling of a wire, wherein the doubling of the wire forms an inverted arch.

15. A method comprising:
positioning a docking device near a sinotubular junction of a patient, wherein the docking device comprises a lattice forming a tubular framework and a plurality of struts attached around the lattice;
expanding the lattice; and
locking an aortic valve replacement inside the docking device.

16. The method of claim 15, wherein the plurality of struts comprises at least three struts configured such that the upper or proximal part of each of the plurality of struts is shaped longitudinally as a curve protruding inward relative to the tubular framework and flattening out toward an upmost end of the upper or proximal part, and the lower or distal part of each of the plurality of struts is shaped longitudinally as a curve protruding outward relative to the tubular framework, and wherein positioning the docking device near the sinotubular junction of the patient comprises:
positioning an upper or proximal part of each of the plurality of struts in an ascending aorta of the patient; and
positioning a lower or distal part of each of the plurality of struts in a Sinus of Valsalva of the patient.

17. The method of claim 16, wherein positioning the docking device near the sinotubular junction of the patient further comprises resting a lowermost end of the lower or distal part of each of the plurality of struts in aortic cusps.

18. The method of claim 17 further comprising expanding the upper or proximal part of each of the plurality of struts by introducing the aortic valve replacement inside the docking device, wherein expanding the upper or proximal part of each of the plurality of struts causes the lower or distal part of each of the plurality of struts to move inward such that aortic valve leaflets are pinched between the docking device and the aortic valve replacement.

19. The method of claim 18, wherein the at least three struts are further configured such that the upper or proximal part of each of the plurality of struts is attached to the lattice, the lower or distal part of each of the plurality of struts is not attached to the lattice, and the lower or distal part of each of the plurality of struts comprises an elongated loop formed by a doubling of a wire, the doubling of the wire forming an inverted arch.

20. The method of claim 19, wherein the lowermost end of the lower or distal part of each of the plurality of struts is shaped transversely as a curve protruding inward relative to the tubular framework.

21. The method of claim 20 wherein the lattice comprises inner protrusions sized to interlock with the aortic valve replacement, and wherein locking the aortic valve replacement inside the docking device is performed using the inner protrusions.
